(19) United States
(12) Patent Application Publication
(10) Pub. No.: US 2012/0289899 A1
(43) Pub. Date: Nov. 15, 2012

(54) SELF-LOCKING SINGLE USE SYRINGE

(76) Inventor: Ming-Yuan WU, Taipei City (TW)

(21) Appl. No.: 13/218,269

(22) Filed: Aug. 25, 2011

(30) Foreign Application Priority Data
May 12, 2011 (TW) 100116590

(51) Int. Cl. A61M 5/50

(52) U.S. Cl. 604/110

(57) ABSTRACT

A self-locking single use syringe comprising a plunger arm, plunger tip, syringe body, and locking sleeve is disclosed. The locking sleeve surrounds tip retaining arms of the plunger arm allowing the tip retaining arms to grasp the plunger tip. When the plunger arm is pulled backwards, sleeve flanges of the locking sleeve protrude into a sleeve lock of the syringe body and the locking sleeve is prevented from moving in a forward direction. As the plunger arm is pushed forward, the tip retaining arms are no longer surrounded by the locking sleeve and the tip retaining arms move outwards and release the plunger tip. After the plunger tip has been released from the plunger arm, the plunger tip and the plunger arm cannot be reconnected and the self-locking single use syringe cannot be reused.

### BACKGROUND OF THE INVENTION

1. Field of the Invention

The present invention relates to medical syringes. More specifically, the present invention discloses a safe and effective syringe that automatically ensures the syringe cannot be reused.

2. Description of the Prior Art

Syringes are commonly used medical implements for injecting medicine into a patient. A medical professional draws medicine into the syringe and then injects the medicine into a patient through a needle in the syringe.

While useful, the conventional syringe has various disadvantages that permit potentially dangerous use of the syringe. Misuse of the traditional syringe can be fatal or detrimental to a patient's health.

One major disadvantage of the conventional syringe is that the syringe can easily be used again. The conventional syringe or components of the syringe can easily be used again since the conventional syringe provides no inherent means to prevent reuse.

After a patient is injected with a medicine, residue such as blood, medicine, virus, bacteria, or other contaminants remain on the needle or inside the syringe. If another patient is injected using the used syringe, the second patient can be contaminated with residue from the first patient.

If the first patient is infected with a virus, the second patient is susceptible to contracting the virus from the previously used syringe. The second patient is also susceptible if the second patient is allergic to the medicine used with the first patient.

Additionally, it can be difficult to determine whether or not the syringe has been previously used.

Therefore, there is need for an improved medical syringe that effectively delivers medicine to a patient while automatically preventing reuse of the syringe.

### SUMMARY OF THE INVENTION

To achieve these and other advantages and in order to overcome the disadvantages of the conventional method in accordance with the purpose of the invention as embodied and broadly described herein, the present invention provides a syringe that automatically locks and disconnects components of the syringe to prevent reuse.

The self-locking single use syringe of the present invention comprises a plunger arm, plunger tip, syringe body, and a locking sleeve. The plunger arm comprises tip retaining arms and an arm post. The arm post is inserted into a mating hollow interior section of a tip base of the plunger tip. The locking sleeve surrounds tip retaining arms of the plunger arm allowing the tip retaining arms to grasp the plunger tip.

When the plunger arm is pulled backwards, sleeve flanges of the locking sleeve protrude into a sleeve lock of the syringe body and the locking sleeve is prevented from moving in a forward direction.

As the plunger arm is pushed forward, the tip retaining arms are no longer surrounded by the locking sleeve and the tip retaining arms move outwards and release the plunger tip. After the plunger tip has been released, the plunger arm can continue to push the plunger tip forward until the end of the syringe body has been reached. However, after the plunger tip has been released, the plunger arm cannot grasp or pull the plunger tip backwards.

After the plunger tip has been released from the plunger arm, the plunger tip and the plunger arm cannot be reconnected and the self-locking single use syringe cannot be reused.

The tip base of the plunger tip further comprises a used indicator. The used indicator comprises, for example, lettering or wording such as “USED” and/or a bright color. After the syringe has been used the used indicator allows easy confirmation that this syringe has been previously used. The used indicator is hidden and not visible until the syringe has been used.

Not only is the self-locking single use syringe of the present invention useful in preventing reuse of the syringe by medical professionals but also prevents addicts or intravenous drug users from sharing needles.

The self-locking single use syringe of the present invention dramatically decreases the need for needle exchange programs to ensure that intravenous drug users do not share needles. Even if the user does not return the used syringe to an exchange center, the self-locking single use syringe cannot be used by another addict.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIG. 1A is a drawing illustrating a cross-sectional view of a plunger actuator of a self-locking single use syringe according to an embodiment of the present invention;

FIG. 1B is a drawing illustrating an end view of a plunger actuator of a self-locking single use syringe according to an embodiment of the present invention;

FIGS. 2A-2B are drawings illustrating a cross-sectional view of a locking sleeve of a self-locking single use syringe according to an embodiment of the present invention;

FIG. 2C is a drawing illustrating an end view of a locking sleeve of a self-locking single use syringe according to an embodiment of the present invention;

FIG. 3A is a drawing illustrating a cross-sectional view of a plunger tip of a self-locking single use syringe according to an embodiment of the present invention;

FIG. 3B is a drawing illustrating an end view of a plunger tip of a self-locking single use syringe according to an embodiment of the present invention;

FIG. 4A is a drawing illustrating a cross-sectional view of a syringe body of a self-locking single use syringe according to an embodiment of the present invention;

FIG. 4B is a drawing illustrating an end view of a syringe body of a self-locking single use syringe according to an embodiment of the present invention;

FIGS. 5A-5H are drawings illustrating movement and operation of a self-locking single use syringe with sleeve stopper and end positioned body grasp flange according to an embodiment of the present invention;

FIGS. 6A-6H are drawings illustrating movement and operation of a self-locking single use syringe with sleeve stopper and mid positioned body grasp flange according to an embodiment of the present invention;

FIGS. 7A-7B are drawings illustrating a cross-sectional view of a locking sleeve of a self-locking single use syringe according to an embodiment of the present invention;

FIG. 7C is a drawing illustrating an end view of a locking sleeve of a self-locking single use syringe according to an embodiment of the present invention;

FIG. 8 is a drawing illustrating a cross-sectional view of a syringe body of a self-locking single use syringe according to an embodiment of the present invention;

FIGS. 9A-9H are drawings illustrating movement and operation of a self-locking single use syringe with end positioned body grasp flange according to an embodiment of the present invention;

FIGS. 10A-10H are drawings illustrating movement and operation of a self-locking single use syringe with mid positioned body grasp flange according to an embodiment of the present invention;

FIG. 11 is a drawing illustrating a plunger arm with plunger flanges according to an embodiment of the present invention;

FIG. 12 is a drawing illustrating a plunger lock and sleeve lock of a syringe body according to an embodiment of the present invention; and

FIGS. 13A-13B are drawings illustrating a plunger tip with tip retaining arms according to embodiments of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

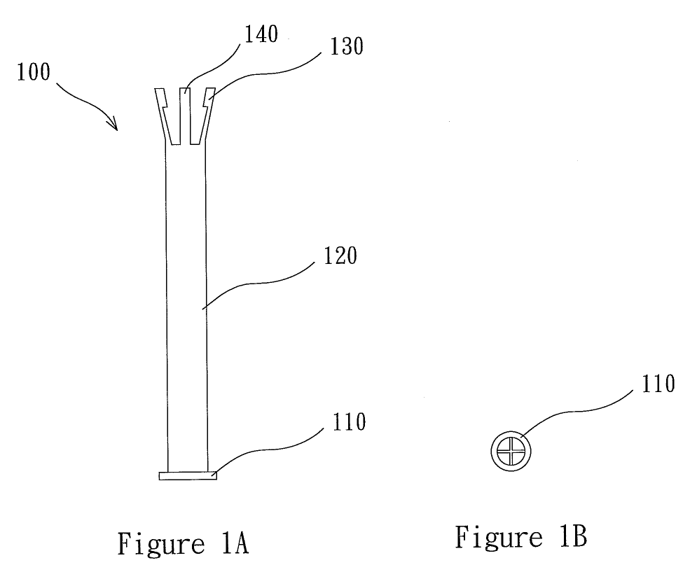

Refer to FIG. 1A, which is a drawing illustrating a cross-sectional view of a plunger actuator of a self-locking single use syringe according to an embodiment of the present invention and to FIG. 1B, which is a drawing illustrating an end view of a plunger actuator of a self-locking single use syringe according to an embodiment of the present invention.

The self-locking single use syringe of the present invention comprises a plunger assembly, a locking sleeve, and a syringe body.

The plunger assembly comprises a plunger arm 100 and a plunger tip that removably attached together. The plunger assembly allows medicine to be drawn into the syringe body and the medicine to be injected into a patient.

As shown in FIGS. 1A and 1B the plunger arm 100 comprises a plunger knob 110, a plunger stem 120, tip retaining arms 130, and an arm post 140.

The plunger knob 110 is positioned on one end of the plunger stem 120. The plunger knob 110 allows a medical professional or user of the syringe to pull back the plunger arm 100 of the plunger assembly in order to draw liquid into the syringe. The plunger knob 110 also allows a professional or user of the syringe to push on the plunger knob 110 in order to move the plunger assembly forward and expel liquid from the syringe.

Positioned on the other end of the plunger stem 120, the plunger arm 100 comprises tip retaining arms 130 and an arm post 140. The tip retaining arms 130 and the arm post 140 cooperate to position and hold the plunger tip of the plunger assembly.

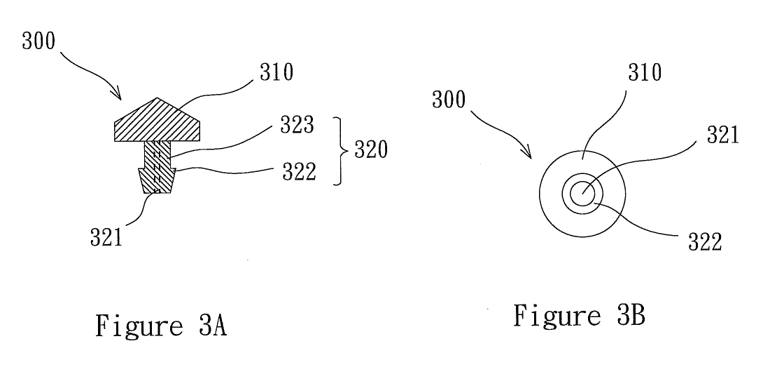

Refer to FIG. 3A, which is a drawing illustrating a cross-sectional view of a plunger tip of a self-locking single use syringe according to an embodiment of the present invention and to FIG. 3B, which is a drawing illustrating an end view of a plunger tip of a self-locking single use syringe according to an embodiment of the present invention.

The plunger assembly of the self-locking single use syringe of the present invention further comprises a plunger tip 300 that connects with the plunger arm.

The plunger tip 300 comprises a tip head 310 and a tip base 320. The tip base 320 comprises a hollow interior area 321 that mates with the arm post (140 FIG. 1A). The tip base 320 further comprises a tab section 322 that allows the tip retaining arms (130 FIG. 1A) to grasp the tip base 320 and hold the plunger tip 300 to the plunger arm (100 FIG. 1A).

In an embodiment of the present invention the hollow interior area 321 and the arm post are round.

In another embodiment of the present invention the hollow interior area 321 and the arm post are formed in a mating non-round shape to prevent the plunger tip 300 from rotating separately from the plunger arm. For example, the arm post and hollow interior area 321 are square, triangle, or diamond shaped. As a result, when the arm post is inserted into the hollow interior area 321 the plunger tip 300 is only allowed to rotate together with the plunger arm.

The tip base 320 further comprises a used indicator 323. The used indicator 323 comprises, for example, lettering or wording such as “USED” and/or a bright color. After the syringe has been used the used indicator 323 allows a user to easily identify that this syringe has been previously used. The used indicator 323 is hidden and not visible until the syringe has been used.

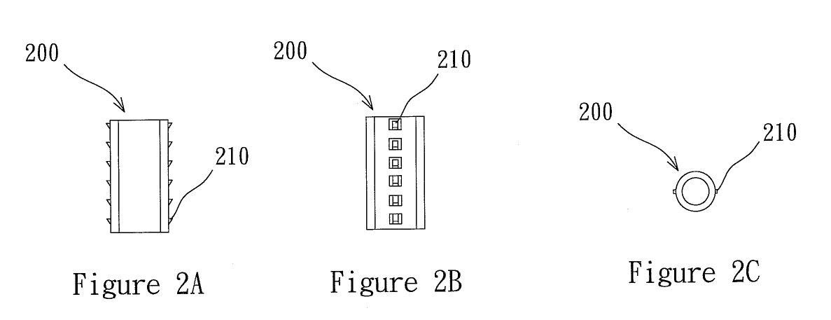

Refer to FIGS. 2A-2B, which are drawings illustrating a cross-sectional view of a locking sleeve of a self-locking single use syringe according to an embodiment of the present invention and to FIG. 2C, which is a drawing illustrating an end view of a locking sleeve of a self-locking single use syringe according to an embodiment of the present invention.

The self-locking single use syringe of the present invention further comprises a locking sleeve 200. The locking sleeve 200 is an elongated tube that is positioned around the plunger arm. A plurality of sleeve flanges 210 are disposed on the locking sleeve 200 and extend outside of the main body of the locking sleeve 200.

In an embodiment of the present invention the locking sleeve 200 is a tube shape.

In another embodiment of the present invention the locking sleeve 200 is split along the length of the locking sleeve for ease of assembly by allowing the locking sleeve to be opened temporarily and wrapped around the plunger arm after which the locking sleeve returns to a closed tube shape.

In an embodiment of the present invention the inside surface of the locking sleeve 200 is angled or has a chamfered end to allow the tip retaining arms to exit the locking sleeve more easily and release the plunger tip sooner.

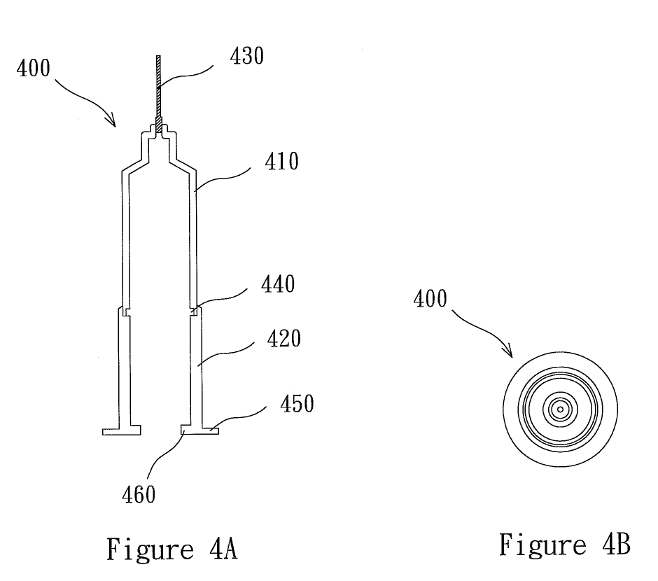

Refer to FIG. 4A, which is a drawing illustrating a cross-sectional view of a syringe body of a self-locking single use syringe according to an embodiment of the present invention and to FIG. 4B, which is a drawing illustrating an end view of a syringe body of a self-locking single use syringe according to an embodiment of the present invention.

The self-locking single use syringe of the present invention further comprises a syringe body 400. The syringe body 400 comprises an upper body 420 and a lower body 410. A needle 430 is molded into the lower body 410. The upper body 420 comprises a body grasp flange 450 and a sleeve stopper 460.

The body grasp flange 450 extends outwardly from the upper body 420 to assist a professional or user during operation of the syringe. To aid in pulling back the plunger assembly the syringe user places a finger or fingers on the top side of the body grasp flange 450 while pulling back on the plunger knob. When pressing on the plunger knob to move the plunger assembly forward the syringe user places a finger or fingers on the bottom side of the body grasp flange 450.

The sleeve stopper 460 prevents the locking sleeve (200 FIG. 2A 2B) from exiting the syringe body 400.

In an embodiment of the present invention the upper body 420 and the lower body 410 are formed in two separate pieces and then connected together. The two pieces are held together by, for example, ultrasonic welding or mating connectors that snap together.

A sleeve lock 440 is disposed in the section of the syringe body 400 where the upper body 420 and the lower body 410 connect. The sleeve lock 440 comprises an inset or indented section of the interior of the syringe body 400. As the locking sleeve (200 FIGS. 2A 2B) travels back in the syringe body 400, the sleeve flanges (210 FIGS. 2A 2B) protrude into the sleeve lock 440. The sleeve lock 440 traps the sleeve flanges and prevents the locking sleeve from traveling forward towards the area where the needle 430 is located.

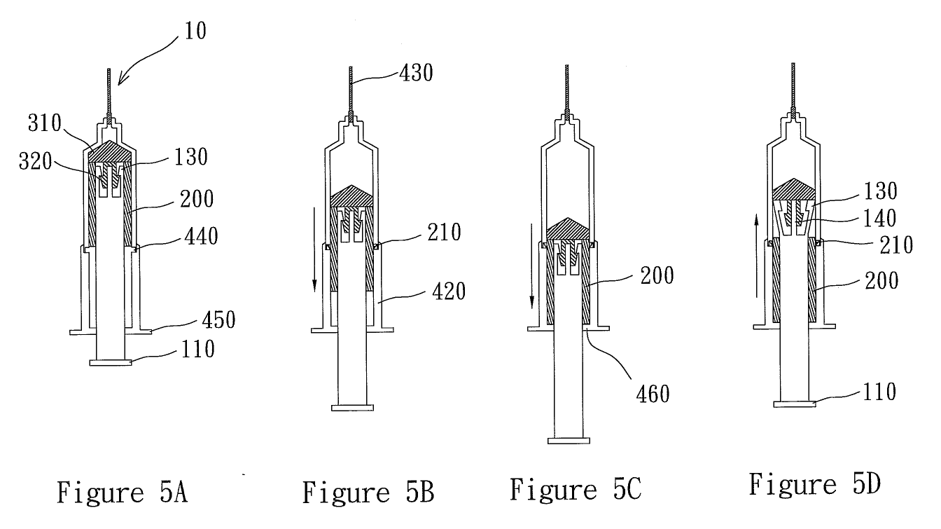

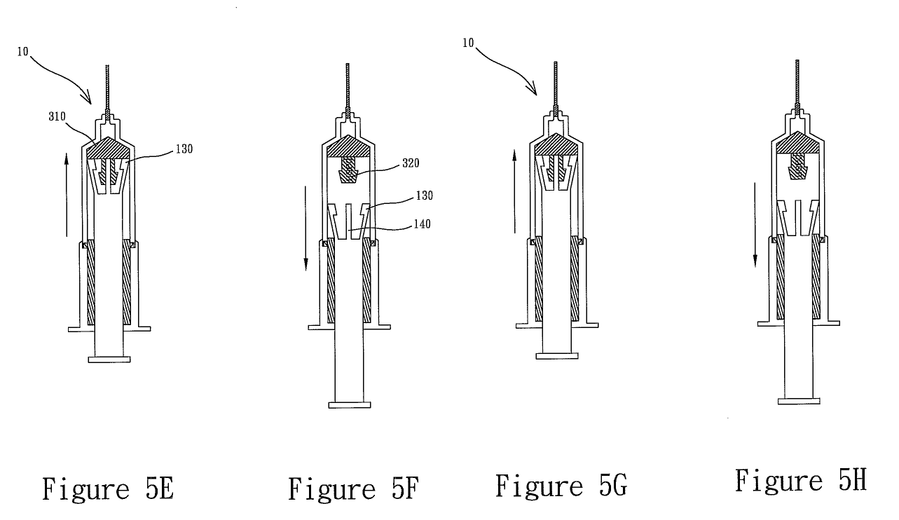

Refer to FIGS. 5A-5H, which are drawings illustrating movement and operation of a self-locking single use syringe with sleeve stopper and end positioned body grasp flange according to an embodiment of the present invention and continue to refer to FIGS. 1A-4B.

When assembled, the self-locking single use syringe 10 of the present invention comprises the plunger assembly (plunger arm 100 and plunger tip 300) and the locking sleeve 200 disposed inside the syringe body 400.

The locking sleeve 200 surrounds a portion of the plunger stern 120 and the tip retaining arms 130. When the locking sleeve 200 surrounds the tip retaining arms 130, the tip retaining arms 130 grasp and hold the tab section 322 of the tip base 320 and the plunger tip 300 is held together with the plunger arm 100.

In an embodiment of the present invention a flange is disposed on the plunger stem 120 to prevent unintentional movement of the locking sleeve 200 during shipping of the self-locking single use syringe 10. However, the flange is appropriately sized and positioned to allow movement of the locking sleeve 200 during intentional use of the syringe 10.

As shown in FIG. 5B, as the plunger arm 100 is pulled back the locking sleeve 200 holds the tip retaining arms 130 in to grasp the tip base 320 so that the plunger tip 300 is pulled back along with the plunger arm 100. As the plunger tip 300 is pulled back the plunger head 310 pulls the locking sleeve 200 back with the plunger tip 300.

As the locking sleeve 200 moves backwards in the syringe body 400, the sleeve flanges 210 of the locking sleeve 200 protrude into the sleeve lock 440 of the syringe body 400. At any time after the first sleeve flange 210 enters the sleeve lock 440, the locking sleeve 200 is prevented from moving in the forward direction. The sleeve flange 210 is formed to allow backwards movement of the locking sleeve 200 and along with the sleeve lock 440 prevent forward movement of the locking sleeve 200. As each successive sleeve flange 210 enters the sleeve lock 440 the locking sleeve 200 is prevented from moving in the forward direction.

As shown in FIG. 5C, as the plunger arm 100 continues to be pulled backward the locking sleeve 200 continues to move backwards along with the plunger assembly (plunger arm 100 and plunger tip 300).

When the locking sleeve 200 reaches the rear opening of the syringe body 400, the sleeve stopper 460 of the upper body 420 blocks the backward movement of the locking sleeve 200 and prevents the locking sleeve from exiting the syringe body 400.

At the position illustrated in FIG. 5C, the locking sleeve 200 is prevented from moving backward by the sleeve stopper 460 and prevented from moving forward by the sleeve flange 210 protruding into the sleeve lock 440.

As shown in FIG. 5D, when the plunger knob 110 is pushed the plunger arm 100 moves forward. Since the locking sleeve 200 is prevented from moving forward, the tip retaining arms 130 move outwards as the locking sleeve 200 no longer surrounds the tip retaining arms 130. After the tip retaining arms 130 have moved outwards and away from the tip base 320, the tip retaining arms 130 no longer grasp the tab section 322 of the tip base 320. However, the plunger tip 300 can continue to be moved in a forward direction since it is now pushed by the arm post 140 and the ends of the tip retaining arms 130.

As shown in FIG. 5E, when the plunger assembly has been moved all the way forward, the end of the lower body 410 of the syringe body 400 stops forward movement of the plunger tip 300.

As shown in FIG. 5F, when the plunger arm 100 is now pulled backwards the plunger tip 300 is separated from the plunger arm 100 since the tip retaining arms 130 no longer grasp the tab section 322 of the tip base 320.

As shown in FIG. 5G and FIG. 5H, after the plunger tip 300 has been released from the grasp of the plunger arm 100, the plunger arm 100 cannot reconnect with the plunger tip 300 and the self-locking single use syringe cannot be used again.

At any time after the tip retaining arms 130 are not surrounded by the locking sleeve and have moved outwards, the plunger tip 300 has been released from the plunger arm 100 and the plunger tip 300 can no longer be pulled backwards by the plunger arm 100.

After the plunger tip 300 has been separated from the plunger arm 100 the used indicator 323 of the tip base 320 provides visual identification and confirmation that the syringe has be used.

Utilizing a plurality of sleeve flanges 210 allows different dosage amounts with a single sized syringe while preventing reuse of the syringe. For example, if the dose requires the plunger assembly to travel backwards 25% of the interior of the syringe body, the sleeve flange 210 prevents the locking sleeve 200 from moving forward and the plunger tip 300 is released from the plunger arm 100. If the dose requires the plunger assembly to travel backwards 50% of the interior of the syringe body, a successive sleeve flange 210 prevents the locking sleeve 200 from moving forward and the plunger tip 300 is released from the plunger arm 100.

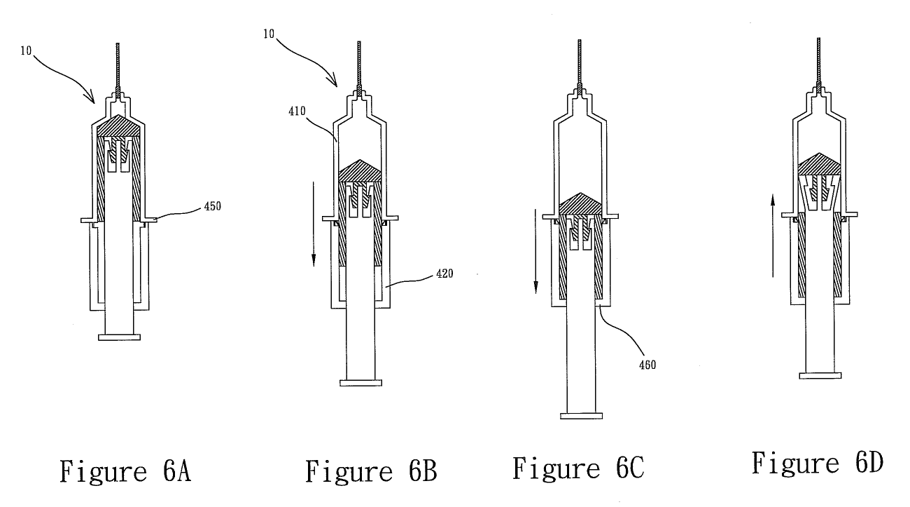

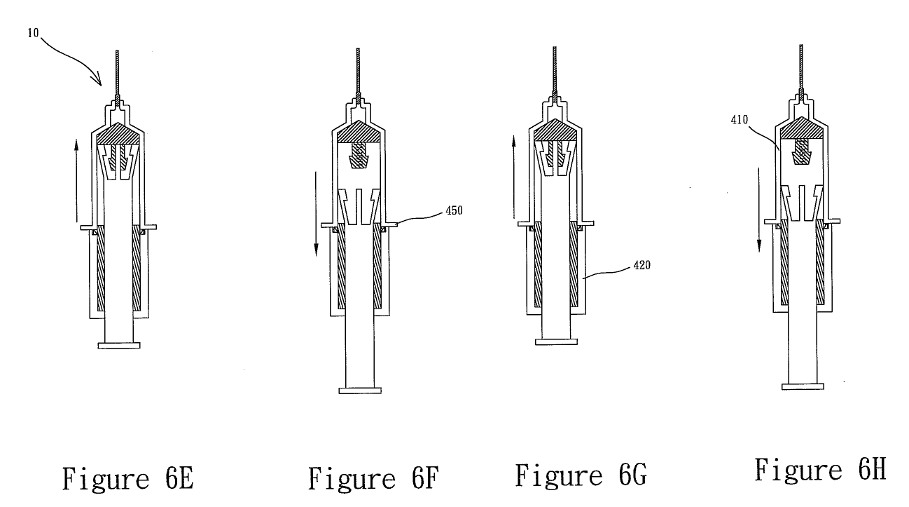

Refer to FIGS. 6A-6H, which are drawings illustrating movement and operation of a self-locking single use syringe with sleeve stopper and mid positioned body grasp flange according to an embodiment of the present invention and continue to refer to FIGS. 1A-4B.

The embodiment illustrated in FIGS. 6A-6H is similar to the embodiment illustrated in FIGS. 5A-6H and the basic operations of the self-locking single use syringe 10 are similar.

However, in the embodiment illustrated in FIGS. 6A-6H the body grasp flange 450 is formed on the lower body 410 of the syringe body 400. With the body grasp flange 450 disposed towards the middle of the syringe body 400, the syringe body 400 is made stronger.

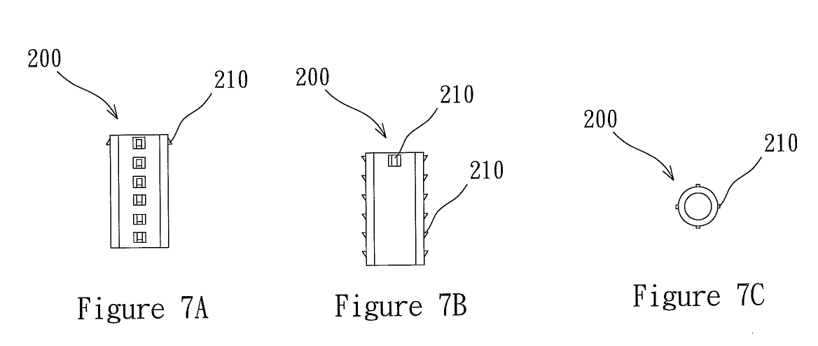

Refer to FIGS. 7A-7B, which are drawings illustrating a cross-sectional view of a locking sleeve of a self-locking single use syringe according to an embodiment of the present invention and to FIG. 7C, which is a drawing illustrating an end view of a locking sleeve of a self-locking single use syringe according to an embodiment of the present invention.

In embodiments of the present invention different formations of the sleeve flanges 210 are provided.

For example, in the embodiment illustrated FIG. 7C the locking sleeve 200 comprises sleeve flanges 210 disposed on four sides of the locking sleeve 200. In the embodiment illustrated in FIG. 2C the locking sleeve 200 comprises sleeve flanges 210 disposed on two sides of the locking sleeve 200.

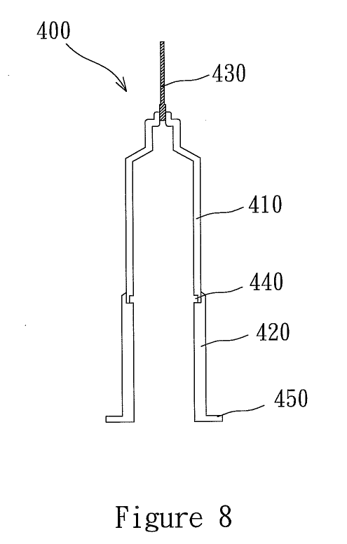

Refer to FIG. 8, which is a drawing illustrating a cross-sectional view of a syringe body of a self-locking single use syringe according to an embodiment of the present invention.

As shown in FIG. 8, in an embodiment of the present invention the syringe body 400 does not comprise a sleeve stopper (460 FIG. 4A) to prevent the locking sleeve from exiting the syringe body 400.

Since the locking sleeve can exit the syringe body 400 the plunger assembly can be pulled backward farther to draw more medicine into the syringe body 400.

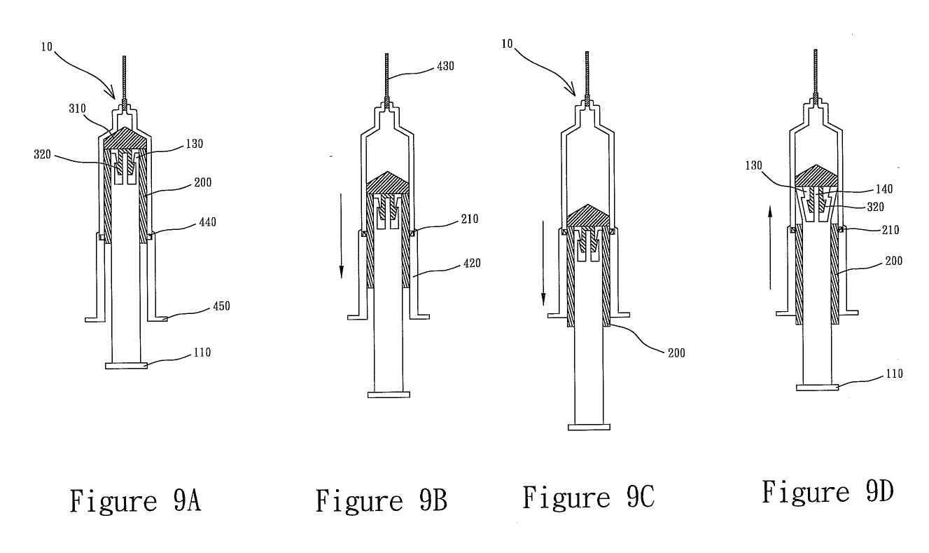

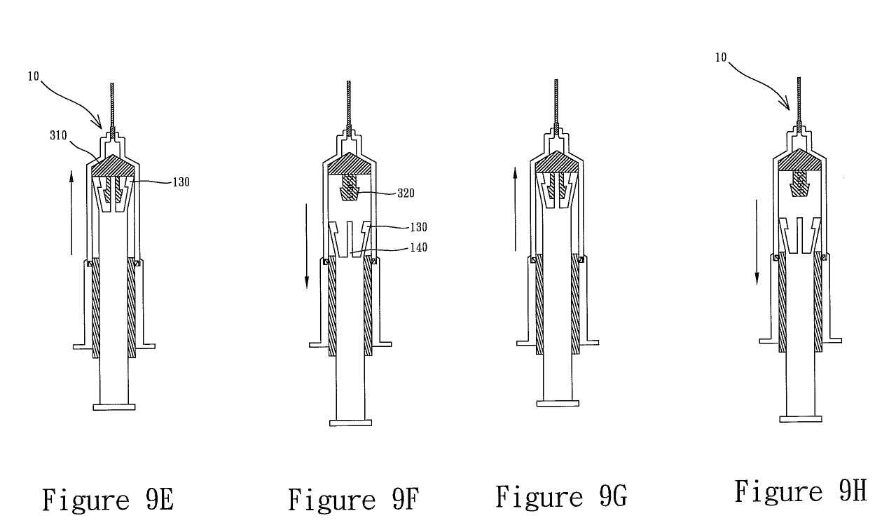

Refer to FIGS. 9A-9H, which are drawings illustrating movement and operation of a self-locking single use syringe with end positioned body grasp flange according to an embodiment of the present invention and continue to refer to FIGS. 1A-4B and 7A-8.

As detailed previously, the locking sleeve 200 surrounds the tip retaining arms 130 and is pulled backward by the plunger assembly (plunger arm 100 and plunger tip 300). However, in this embodiment as shown in FIG. 9C the absence of a sleeve stopper (460 FIG. 4A) allows the locking sleeve 200 to begin to exit the rear opening of the syringe body 400.

The locking sleeve 200 is pulled backwards by the plunger assembly until the desired amount of medicine or liquid has been drawn into the syringe body 100.

When the plunger arm is pushed forward, the sleeve flange 210 protruding into the sleeve lock 440 prevents the locking sleeve 200 from moving forward. Since the locking sleeve 200 no longer surrounds the tip retaining arms 130, the tip retaining arms 130 move outwards and the plunger tip 300 is released from the plunger arm 100.

As shown in FIGS. 9E-9H, after the plunger tip 300 has been released from the plunger arm 100, the plunger tip 300 can no longer be reconnected with or be pulled backwards by the plunger arm 100.

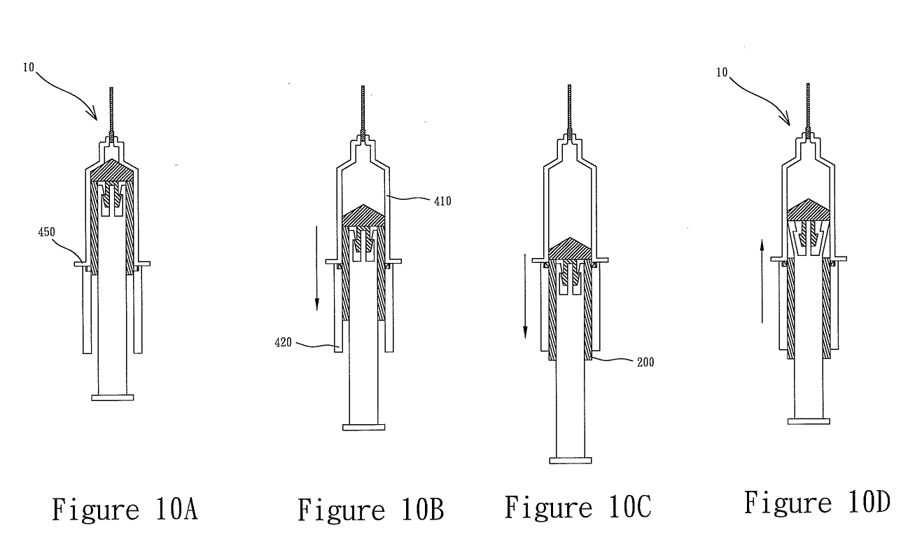

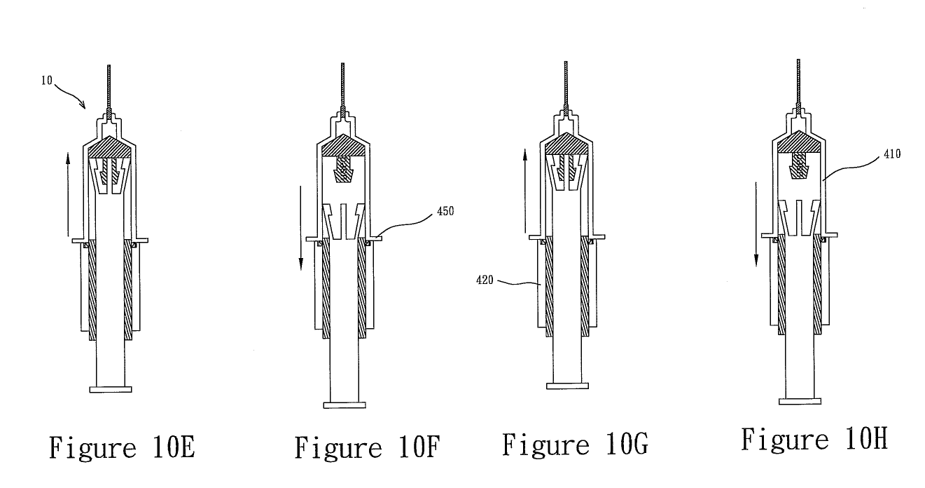

Refer to FIGS. 10A-10H, which are drawings illustrating movement and operation of a self-locking single use syringe with mid positioned body grasp flange according to an embodiment of the present invention and continue to refer to FIGS. 1A-4B and 7A-8.

The operation of the self-locking single use syringe illustrated in FIGS. 10A-10H is similar to the embodiment illustrated in FIGS. 9A-9H. However, in the embodiment illustrated in FIGS. 10A-10H, the body grasp flange 450 is formed towards the middle section of the syringe body 400.

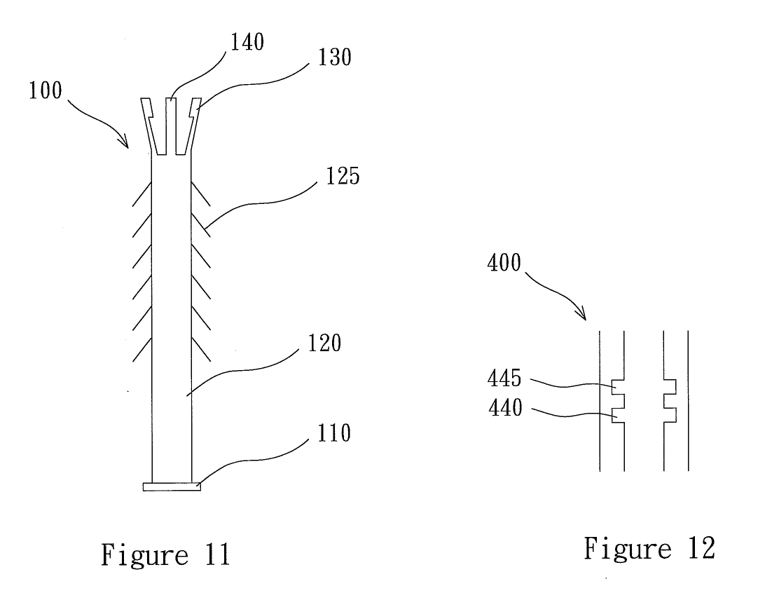

Refer to FIG. 11, which is a drawing illustrating a plunger arm with plunger flanges according to an embodiment of the present invention and to FIG. 12, which is a drawing illustrating a plunger lock and sleeve lock of a syringe body according to an embodiment of the present invention.

In the embodiment illustrated in FIG. 11, the plunger arm 100 comprises a plurality of plunger flanges 125 disposed along the plunger stem 120.

In the embodiment illustrated in FIG. 12, the syringe body 400 comprises a sleeve lock 440 and a plunger lock 445.

After the locking sleeve has been trapped in the sleeve lock 440 and the plunger arm 100 is moved forward the plurality of plunger flanges 125 exit the locking sleeve. The plunger flanges 125 allow forward movement of the plunger arm 100 but prevent backward movement of the plunger arm 100. If the plunger arm 100 is pulled back the plunger flanges 125 are trapped in the indentation of the plunger lock 445. Locking the plunger arm 100 using the plunger flanges 125 and the plunger lock 445 further prevents reuse of the self-locking single use syringe of the present invention.

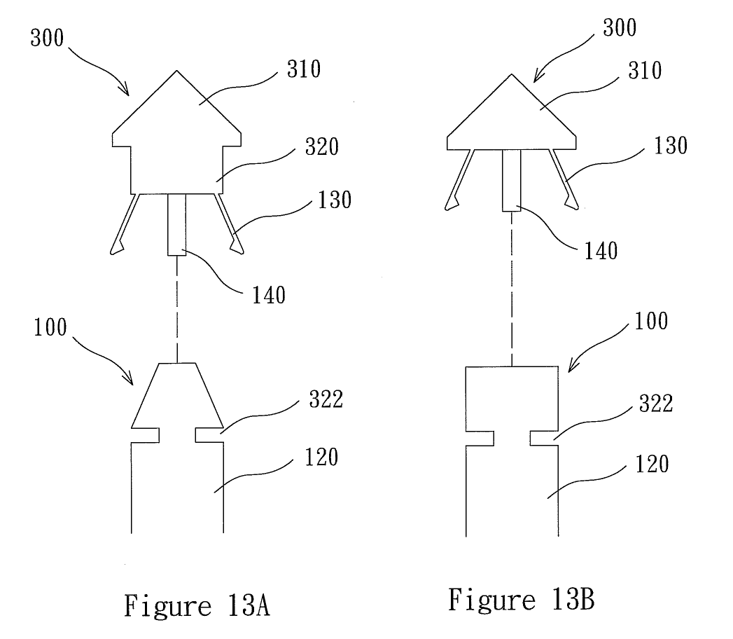

Refer to FIGS. 13A-13B, which are drawings illustrating a plunger tip with tip retaining arms according to embodiments of the present invention.

In embodiments of the present invention the tip retaining arms 130 are disposed on the tip base 320 and the tab section 322 is disposed on the plunger arm 100. When the locking sleeve 200 surrounds the tip retaining arms 130 of the tip base 320, the tip retaining arms 130 grasp the tab section 322 of the plunger arm and the plunger tip 300 is held to the plunger arm 100. When the plunger arm 100 moves forward the tip retaining arms 130 of the plunger tip 300 move outwards and the plunger tip 300 is released from the plunger arm 100.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. A syringe comprising:
a plunger arm, the plunger arm comprising at least one tip retaining arm;
a plunger tip, the plunger tip removably attached to the plunger arm by the at least one tip retaining arm;
a locking sleeve surrounding the at least one tip retaining arm; and
a syringe body holding the plunger arm, the plunger tip, and the locking sleeve.

2. The syringe of claim 1, the locking sleeve comprising at least one sleeve flange.

3. The syringe of claim 2, the syringe body comprising a sleeve lock for trapping the at least one sleeve flange to prevent forward movement of the locking sleeve.

4. The syringe of claim 1, where the plunger tip remains inside the syringe body before and after use.

5. The syringe of claim 1, where the at least one tip retaining arm releases the plunger tip after exiting the locking sleeve.

6. The syringe of claim 1, the syringe body further comprising:
a sleeve stopper for preventing the locking sleeve from exiting the syringe body.

7. The syringe of claim 1, the syringe body comprising:
a lower body comprising a needle;
an upper body comprising a body grasp flange; and
a sleeve lock formed at a junction of the lower body and the upper body.

8. The syringe of claim 1, the syringe body comprising:
a lower body comprising a needle and a body grasp flange;
an upper body; and
a sleeve lock formed at a junction of the lower body and the upper body.

9. The syringe of claim 3, where the at least one tip retaining arm exits the locking sleeve during forward movement of the plunger arm after the at least one sleeve flange has been trapped by the sleeve stopper.

10. A syringe comprising:
a plunger tip;
a plunger arm, the plunger arm comprising at least one tip retaining arm for grasping the plunger tip;
a locking sleeve comprising at least one sleeve flange, the locking sleeve surrounding the at least one tip retaining arm when the at least one tip retaining arm is grasping the plunger tip; and
a syringe body surrounding the plunger arm, the plunger tip, and the locking sleeve, the syringe body comprising a sleeve lock for trapping the at least one sleeve flange to prevent forward movement of the locking sleeve.

11. The syringe of claim 10, where the plunger tip remains inside the syringe body before and after use.

12. The syringe of claim 10, where the at least one tip retaining arm releases the plunger tip after exiting the locking sleeve.

13. The syringe of claim 10, the syringe body further comprising:
a sleeve stopper for preventing the locking sleeve from exiting the syringe body.

14. The syringe of claim 10, the syringe body comprising:
a lower body comprising a needle; and
an upper body comprising a body grasp flange.

15. The syringe of claim 10, the syringe body comprising:
a lower body comprising a needle and a body grasp flange; and
an upper body.

16. The syringe of claim 10, where the at least one tip retaining arm exits the locking sleeve during forward movement of the plunger arm after the at least one sleeve flange has been trapped by the sleeve stopper.

17. A syringe comprising:
a plunger arm, the plunger arm comprising:
at least one tip retaining arm; and
an arm post;
a plunger tip, the plunger tip comprising:
a tip head; and
a tip base, the tip base comprising a tab section and a hollow interior area, the plunger tip removably attached to the plunger arm by the at least one tip retaining arm grasping the tab section of the tip base;
a locking sleeve surrounding the at least one tip retaining arm when the at least one tip retaining arm is grasping the tab section; and
a syringe body for housing the plunger arm, plunger tip, and the locking sleeve.

18. The syringe of claim 17, the locking sleeve comprising at least one sleeve flange.

19. The syringe of claim 18, the syringe body comprising a sleeve lock for trapping the at least one sleeve flange to prevent forward movement of the locking sleeve.

20. The syringe of claim 19, where the at least one tip retaining arm exits the locking sleeve during forward movement of the plunger arm after the at least one sleeve flange has been trapped by the sleeve stopper.

21. The syringe of claim 17, where the plunger tip remains inside the syringe body before and after use.

22. The syringe of claim 17, where the at least one tip retaining arm releases the plunger tip after exiting the locking sleeve.

23. The syringe of claim 17, the syringe body further comprising:
a sleeve stopper for preventing the locking sleeve from exiting the syringe body.

24. The syringe of claim 17, the syringe body comprising:
a lower body comprising a needle;
an upper body comprising a body grasp flange; and
a sleeve lock formed at a junction of the lower body and the upper body.

25. The syringe of claim 17, the syringe body comprising:
a lower body comprising a needle and a body grasp flange;
an upper body; and
a sleeve lock formed at a junction of the lower body and the upper body.

26. A syringe comprising:
a plunger tip, the plunger tip comprising:
a tip head; and
a tip base, the tip base comprising:
a tab section; and
a hollow interior area;
a plunger arm, the plunger arm comprising:
at least one tip retaining arm for grasping the tab section of the tip base; and
an arm post inserted into the hollow interior area of the tip base;
a locking sleeve comprising at least one sleeve flange, the locking sleeve surrounding the at least one tip retaining arm when the at least one tip retaining arm is grasping the tab section; and
a syringe body surrounding the plunger arm, the plunger tip, and the locking sleeve, the syringe body comprising a sleeve lock for trapping the at least one sleeve flange to prevent forward movement of the locking sleeve.

27. The syringe of claim 26, where the plunger tip remains inside the syringe body before and after use.

28. The syringe of claim 26, where the at least one tip retaining arm releases the tab section after exiting the locking sleeve.

29. The syringe of claim 26, the syringe body further comprising:
a sleeve stopper for preventing the locking sleeve from exiting the syringe body.

30. The syringe of claim 26, the syringe body comprising:
a lower body comprising a needle; and
an upper body comprising a body grasp flange.

31. The syringe of claim 26, the syringe body comprising:
a lower body comprising a needle and a body grasp flange; and
an upper body.

32. The syringe of claim 26, where the at least one tip retaining arm exits the locking sleeve during forward movement of the plunger arm after the at least one sleeve flange has been trapped by the sleeve stopper.
